(19) United States
(12) Patent Application Publication
(10) Pub. No.: US 2016/0326266 A1
(43) Pub. Date: Nov. 10, 2016

(54) Chemically-Locked Bispecific Antibodies

(71) Applicant: Sorrento Therapeutics, Inc., San Diego, CA (US)

(72) Inventors: Yanwen Fu, San Diego, CA (US); Gunnar F. Kaufmann, San Diego, CA (US); Bryan Jones, San Diego, CA (US); Rahaleh Toughiri, San Diego, CA (US)

(73) Assignee: Sorrento Therapeutics, Inc., San Diego, CA (US)

(21) Appl. No.: 14/708,273

(22) Filed: May 10, 2015

(51) Int. Cl. C07K 16/46

(52) U.S. Cl. CPC C07K 16/468; C07K 2317/53; C07K 2317/31

(57) ABSTRACT

There is disclosed a process for forming chemically-locked bispecific or heterodimer antibodies, preferably in the IgG class, in high specificity and with high homogeneity. More specifically, there is disclosed a chemically-locked bispecific IgG class antibody having a linkage region joined together with bio-orthogonal click chemistry.

### TECHNICAL FIELD

The present disclosure provides a process for forming chemically-locked bispecific or heterodimer antibodies, preferably in the IgG class, in high specificity and with high homogeneity. More specifically, the present disclosure provides a chemically-locked bispecific IgG class antibody having a linkage region joined with bio-orthogonal click chemistry.

### BACKGROUND

Human immunoglobulin G or IgG antibodies exist in four subclasses, each with distinct structural and functional properties. IgGs are composed of two heavy chain-light chain pairs (half-antibodies) which are connected via inter-heavy chain disulfide bonds directly linking Cys residues in the hinge region (EU-index numbering: cysteine residues 226 and 229; Kabat numbering: cysteine residues 239 and 242). Human IgG4 molecules exist in various molecular forms which differ by the absence or presence of inter-heavy chain disulfide bonds.

A wide variety of recombinant antibody formats have been developed, such as, tetravalent bispecific antibodies by fusion of an IgG antibody format and single chain domains (Coloman et al., Nature Biotech 15 (1997) 159-163; WO 2001/077342; and Morrison, Nature Biotech 25 (2007) 1233-1234). Another format has the antibody core structure (IgA, IgD, IgE, IgG or IgM) no longer retained, such as dia-, tria- or tetrabodies, minibodies, several single chain formats (scFv, Bis-scFv). But such formats are capable of binding two or more antigens (Holliger et al., Nature Biotech 23 (2005) 1126-1136; Fischer and Leger, Pathobiology 74 (2007) 3-14; Shen et al., J. Immunological Methods 318 (2007) 65-74; and Wu et al., Nature Biotech. 25 (2007) 1290-1297).

A method for separating or preferentially synthesizing dimers which are linked via at least one interchain disulfide linkage from dimers which are not linked via at least one interchain disulfide linkage from a mixture comprising the two types of polypeptide dimers is reported in US 2005/0163782.

Bispecific antibodies have difficulty producing materials in sufficient quantity and quality using traditional hybrid hybridoma and chemical conjugation methods. Further, WO2005/062916 and U.S. patent application 2010/0105874 describe how to form bispecific antibodies by reducing antibody “AA” and antibody “BB” to separate the disulfide bonds into single heavy-light chain units (A or B) with a single binding region (wherein both A and B bind to different targets). And then allowing the disulfide bonds to undergo isomerization such that antibodies AB, BA, AA and BB are reformed at a probability of about 25% each. However, both AB and BA are the same bispecific antibodies and therefore represent, at best, about a 50% yield. Therefore, this requires additional steps to separate the desired bispecific antibodies formed from the original reconstituted antibodies. However, U.S. patent application 2010/0105874 points to the hinge region in IgG4 having a sequence of CPSC and stating: “the CPSC sequence results in a more flexible core hinge and the possibility to form intra-chain disulfide bonds . . . it is believed that antibodies having an IgG4-like core hinge sequence may have an intrinsic activity for rearrangement of disulfide bonds, which is simulated by the conditions used in the methods of the invention.” (paragraph 0013). In addition, other forms of bispecific antibodies have been made with a “knob and hole” structure made by altering the sequence of the heavy chains of antibodies A and B.

Therefore, the present disclosure provides a process to produce chemically-locked bispecific IgG antibodies that address the need in the art for a much higher yield of bispecific antibodies and with better stability than the knob and hole methods that alter amino acid sequences in the fixed antibody regions.

### SUMMARY

The present disclosure provides a process for generation of a chemically-locked bispecific antibody “AB” or “BA” from IgG class antibody “A” and IgG class antibody “B” comprising:

(a) reducing an antibody “A” with the hinge residue sequence (EU-index numbering: residues 226-229; Kabat numbering: residues 239-242) CPPC or CPSC or SPPC or SPSC and a second antibody “B” with the hinge residue sequence (residues 226-229) CPPC or CPSC or SPPC or SPSC to form half-antibody A and half-antibody-B, whereby the reducing conditions break any inter-chain or intra-chain disulfide bonds in a hinge region of antibody with the hinge residue sequence (EU-index numbering: residues 226-229; Kabat numbering: residues 239-242) CPPC or CPSC or SPPC or SPSC;

(b) linking a compound selected from the group consisting of:

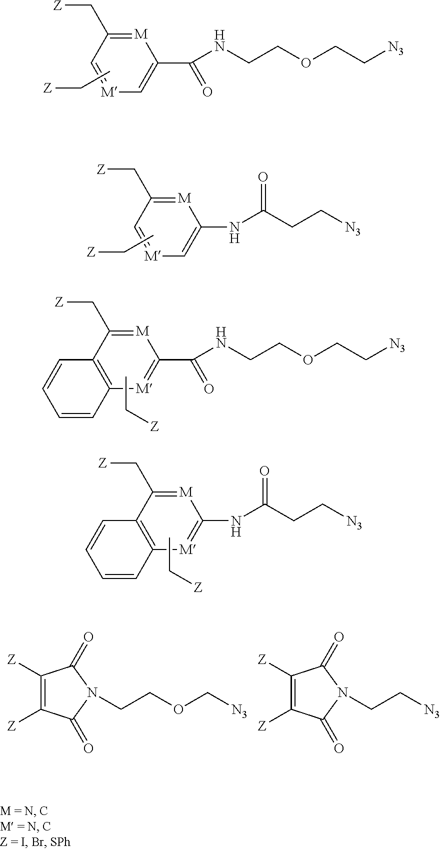

wherein N₃ is —N═N═N;
to one or both Cys residues (EU-index numbering: residues 226 and 229; Kabat numbering: residues 239 and 242) of the hinge core sequence of half-antibody A to form a linked half-antibody A;

(c) linking a compound selected from the group consisting of:

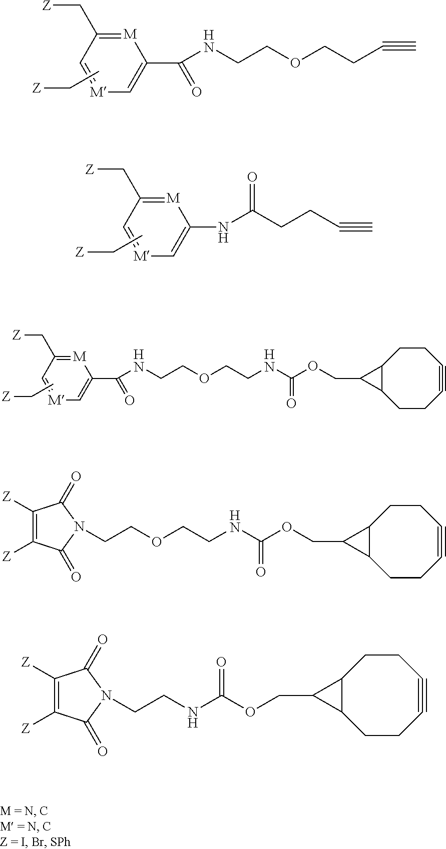

to one or both Cys residues 226 and 229 (EU-index numbering: residues 226 and 229; Kabat numbering: residues 239 and 242) of the hinge core sequence of antibody B to form a linked antibody B; and

(d) incubating approximately equal molar amounts of linked antibody A with linked antibody B under acidic conditions to form the bispecific antibody AB that are linked.

Preferably, the reduction of antibody A to form half-antibody A and antibody B to form half-antibody B is conducted in a reducing agent, wherein the reducing agent is selected from the group consisting of L-cysteine, dithiothreitol, beta-mercapto ethanol, cysteamine, TCEP (tris(2-carboxyethyl)phosphine), 2-MEA (2-Mercaptoethylamine), and combinations thereof. Preferably the hinge region of antibody A, having one or two Cys residues, is linked with a moiety A having the structure selected from the group consisting of:

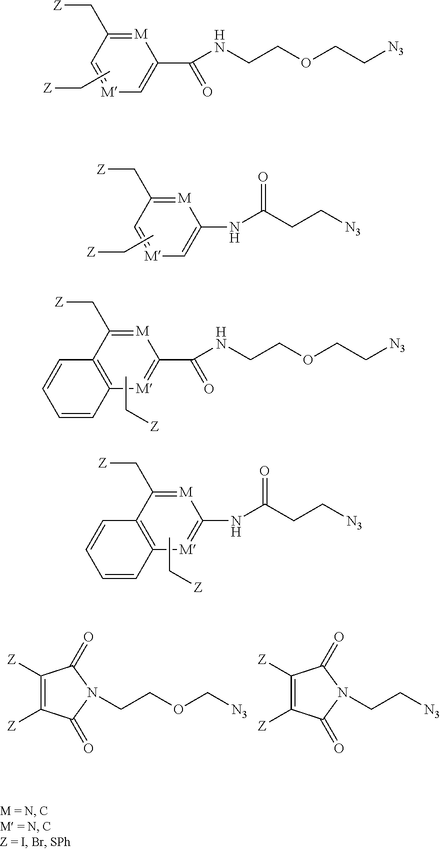

wherein N₃ is —N═N═N. Preferably the hinge region of antibody B, having one or two Cys residues, is linked with a moiety B having the structure selected from the group consisting of:

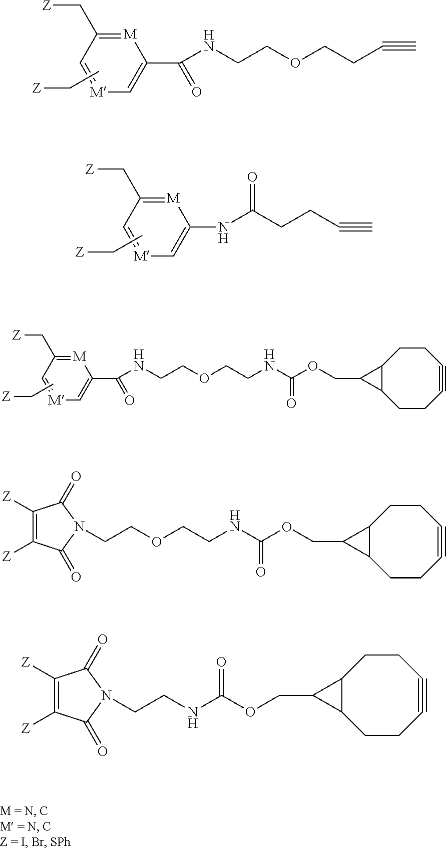

to form a linked half-antibody A.

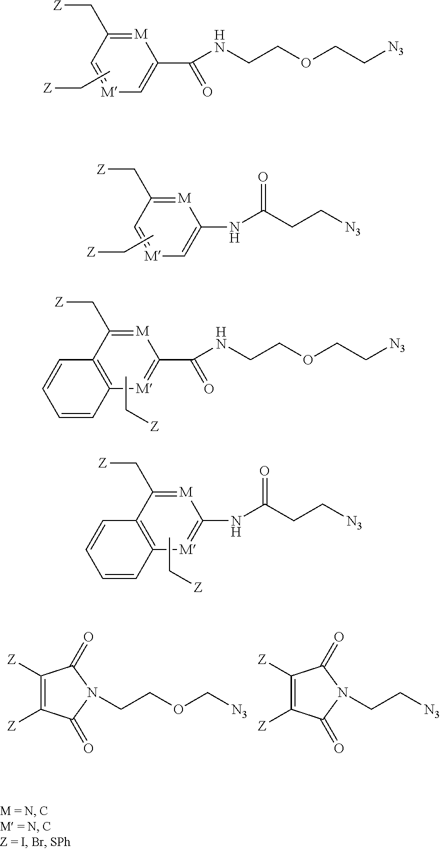

wherein N₃ is —N═N═N;

and a linked antibody B having the structure selected from the group consisting of:

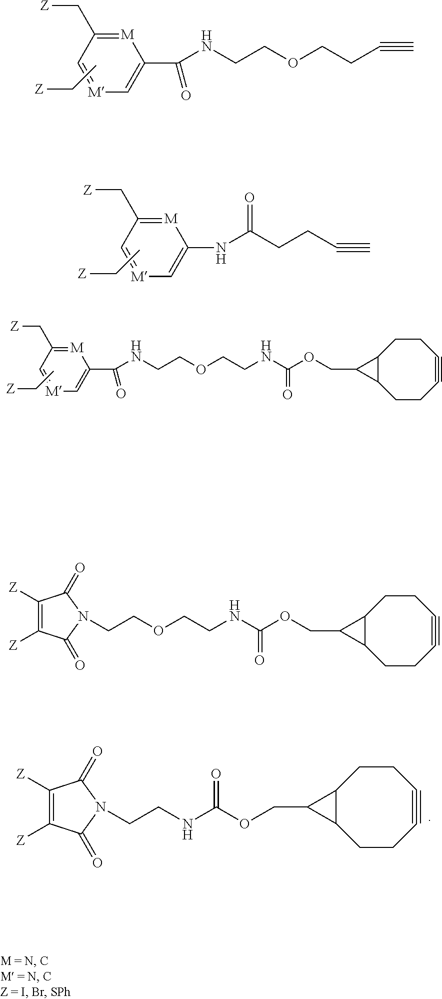

The present disclosure further provides a chemically-locked bispecific antibody AB, wherein a linked half-antibody A

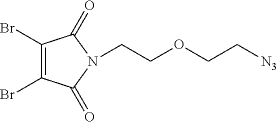

wherein N₃ is —N═N═N;
joins a linked antibody B

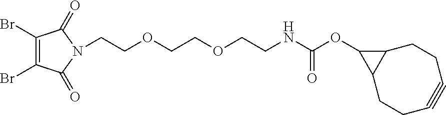 [SMILES: O=C(NCCOCCOCCN1C(=O)C(Br)=C(Br)C1=O)OC1C2CCC#CCCC21]

to form a bispecific antibody AB having the structure shown in FIGS. 5 and 6.

The present disclosure provides a chemically-locked bispecific antibody “AB” or “BA” from IgG class antibody “A” and IgG class antibody “B” comprising a half-antibody A having a structure selected from the group consisting of:

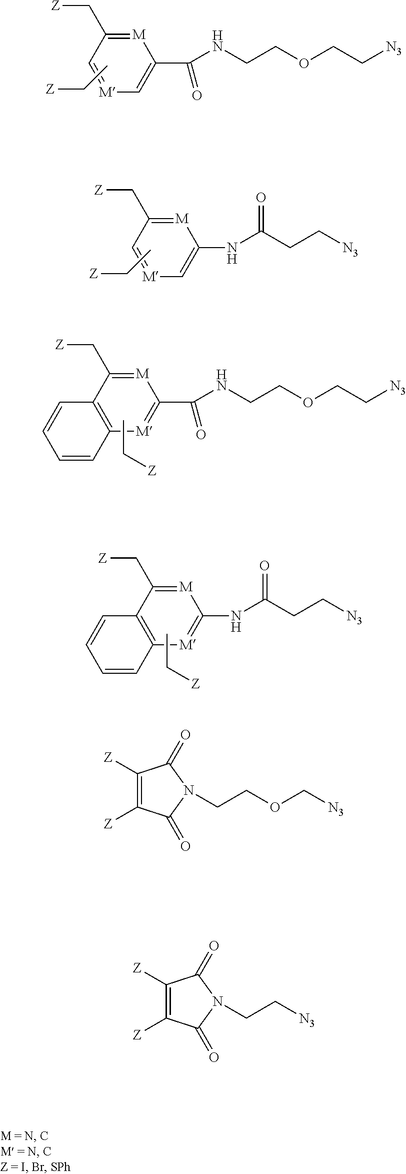

wherein N₃ is —N═N═N, and wherein Z is the leaving group that binds to;

and a half-antibody B having the structure selected from the group consisting of:

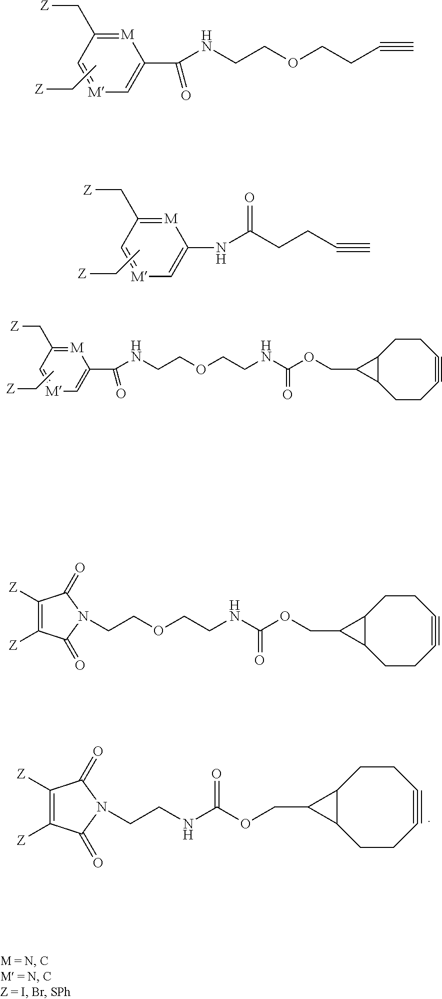

### BRIEF DESCRIPTION OF THE DRAWINGS

FIG. 1 shows a schematic illustration setting up the generation of bispecific mAb via chemical conjugation to a single Cys residue in the hinge region of an IgG class antibody.

FIG. 2 shows a schematic representation of inter-chain cross-link via chemical conjugation to a single Cys residue in the hinge region of an IgG class antibody according to the disclosure herein.

FIG. 3 shows a schematic representation of an intra-chain cross link to two Cys residues within the hinge region of an IgG class antibody.

FIG. 4 shows (top and bottom) a schematic representation for generation of bispecific mAb via inter-chain cross linking to two Cys residues within the hinge region of an IgG class antibody.

FIG. 5 shows an SDS PAGE analysis of chemically locked half-mAb fragments.

FIG. 6 shows an MS analysis of HC Fab from naked mAb (top), azide-conjugated mAb fragment (middle) and alkyne-conjugated mAb fragment (bottom).

FIG. 7 shows SDS PAGE of cross-link products from azide-attached half mAb and alkyne-attached half mAb fragments.

FIG. 8 shows MS analysis of (Fab)₂ from starting mAb (top) and cross-link products (bottom).

### DETAILED DESCRIPTION

The present disclosure provides a process for generation of a chemically-locked bispecific antibody “AB” or “BA” from IgG class antibody “A” and IgG class antibody “B” comprising:

(a) reducing a first antibody “A” with the hinge residue sequence (EU-index numbering: residues 226-229; Kabat numbering: residues 239-242) CPPC or CPSC or SPPC or SPSC and a second antibody “B” with the hinge residue sequence (EU-index numbering: residues 226-229; Kabat numbering: residues 239-242) CPPC or CPSC or SPPC or SPSC to form half-antibody A and half-antibody-B, wherein antibody A binds to a first target and antibody B binds to a second target, whereby the reducing conditions break any inter-chain or intra-chain disulfide bonds in a hinge region of an class antibody with the hinge residue sequence (residues 226-229) CPPC or CPSC or SPPC or SPSC;

(b) linking a compound from formula I to one or two Cys residues (EU-index numbering: residues 226 and 229; Kabat numbering: residues 239 and 242) of the hinge core sequence of half-antibody A to form a linked half-antibody A having a structure selected from the group consisting of:

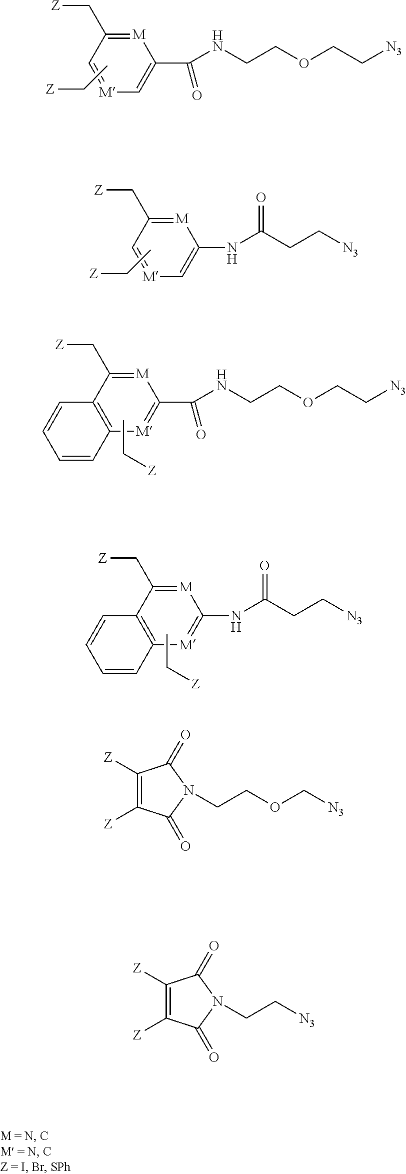

wherein N₃ is —N═N═N;

(c) linking a compound from formula II to one or two Cys residues (EU-index numbering: residues 226 and 229; Kabat numbering: residues 239 and 242) of the hinge core sequence of antibody B to form a linked antibody B having the structure selected from the group consisting of:

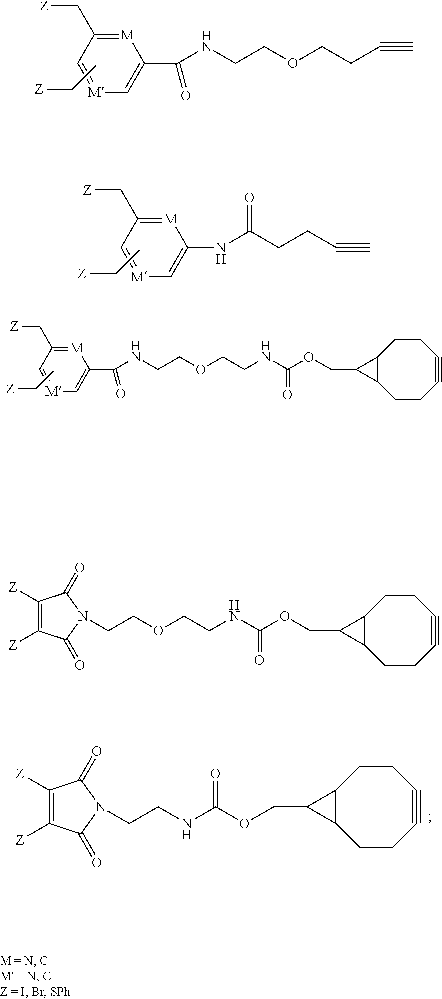

and

(d) incubating approximately equal molar amounts of linked antibody A with linked antibody B under acidic conditions to form the bispecific antibody AB that are linked.

Preferably, the reduction of antibody A to form half-antibody A and antibody B to form half-antibody B is conducted in a reducing agent, such as L-cysteine, dithiothreitol, beta-mercapto ethanol, cysteamine, TCEP (tris(2-carboxyethyl)phosphine), 2-MEA (2-Mercaptoethylamine), and combinations thereof. Preferably the hinge region of antibody A, having two Cys residues, is linked with a moiety A having the structure selected from the group consisting of:

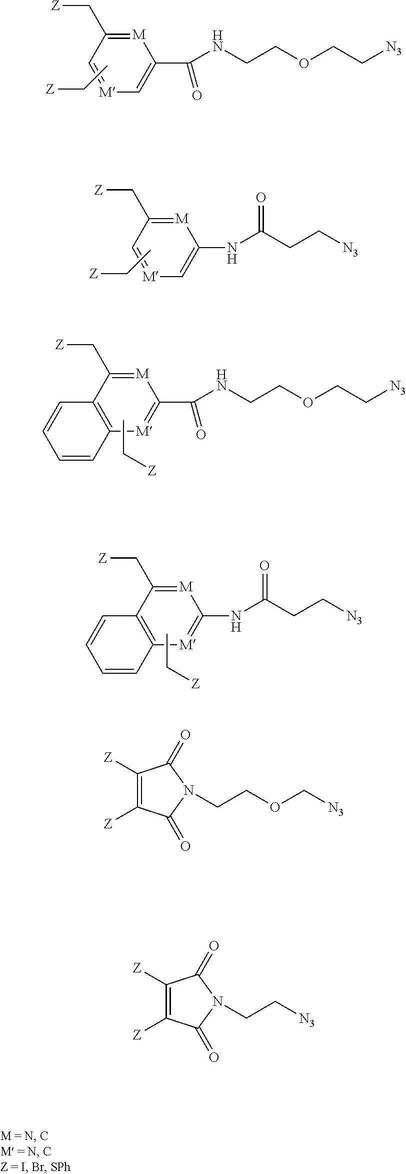

wherein N₃ is —N═N═N. Preferably the hinge region of antibody B, having two Cys residues, is linked with a moiety B having the structure selected from the group consisting of:

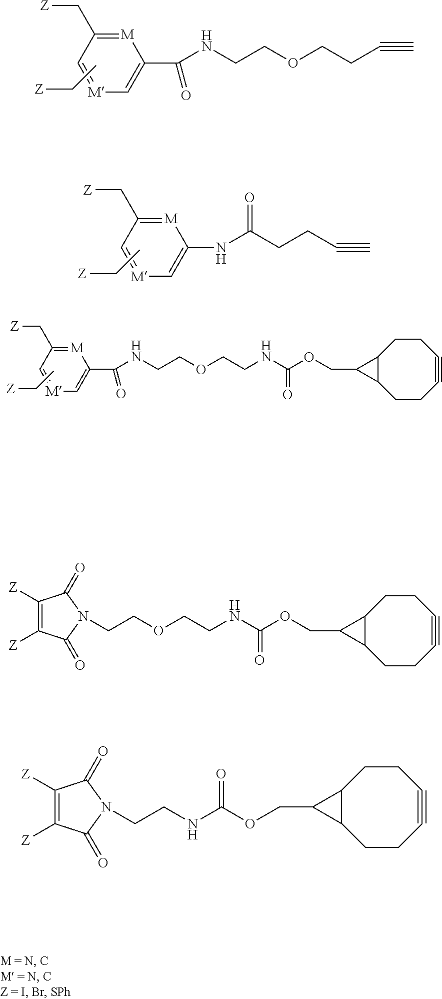

to form a linked half-antibody A having a structure selected from the group consisting of:

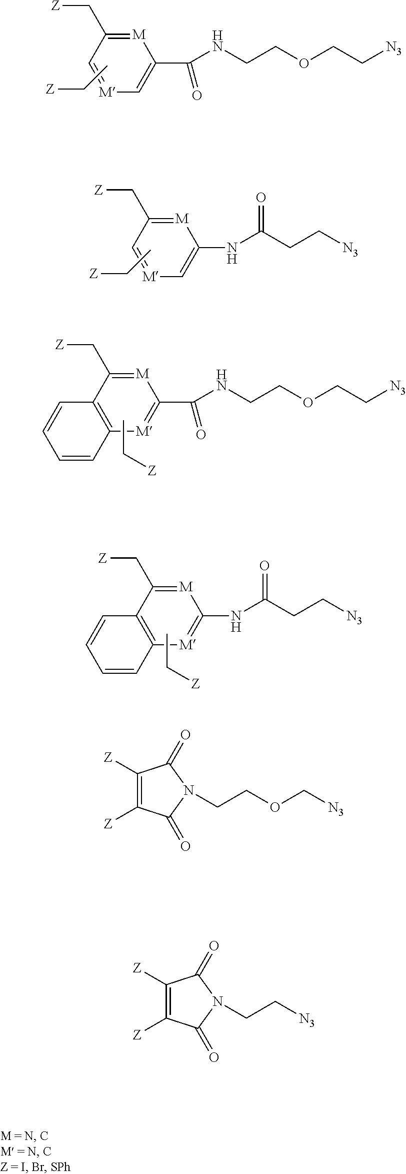

wherein N₃ is —N═N═N;

and a linked antibody B having the structure selected from the group consisting of:

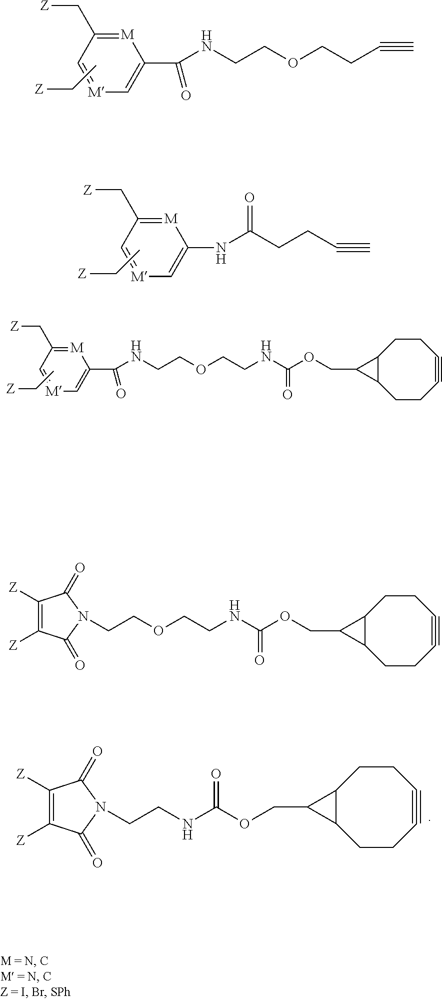

The present disclosure further provides a chemically-locked bispecific antibody AB, wherein a linked half-antibody A

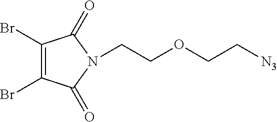

wherein N₃ is —N═N═N;
joins a linked antibody B

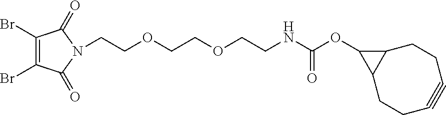 [SMILES: O=C(NCCOCCOCCN1C(=O)C(Br)=C(Br)C1=O)OC1C2CCC#CCCC21]

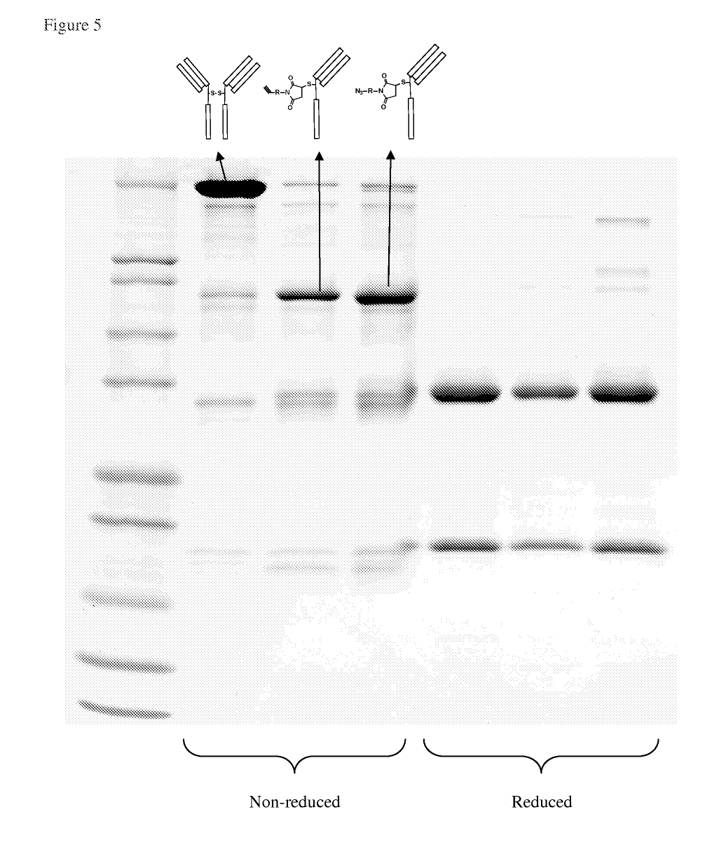

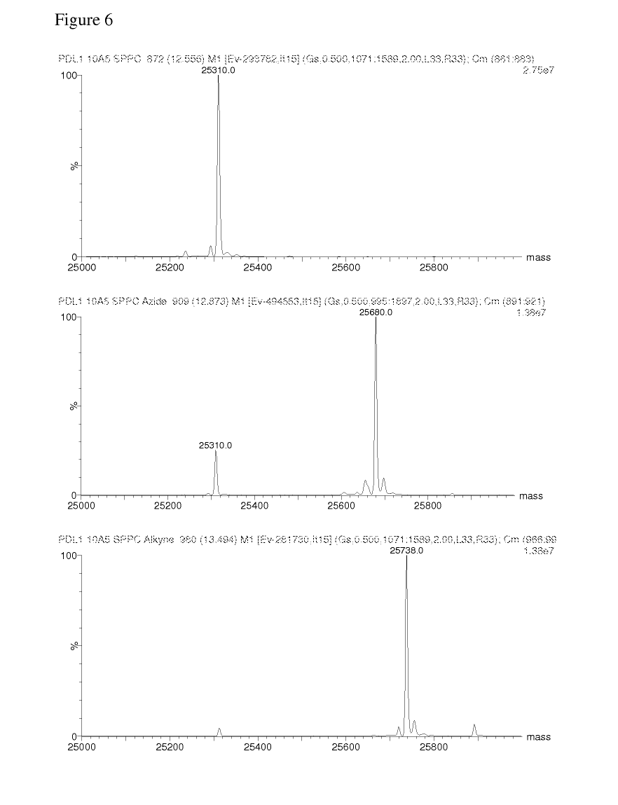

to form a bispecific antibody AB having the structure shown in FIGS. 5 and 6.

The present disclosure provides a chemically-locked bispecific antibody “AB” or “BA” from IgG class antibody “A” and IgG class antibody “B” comprising a half-antibody A having a structure selected from the group consisting of:

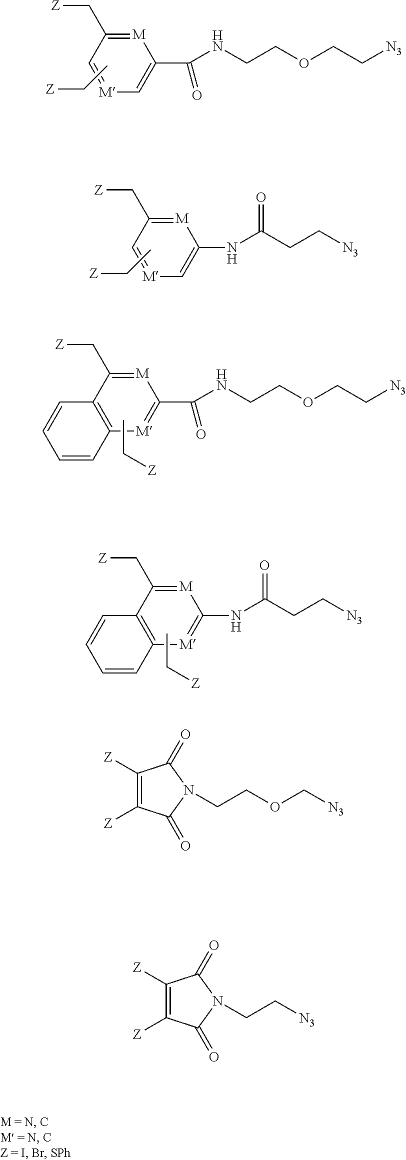

wherein N₃ is —N═N═N;

and a half-antibody B having the structure selected from the group consisting of:

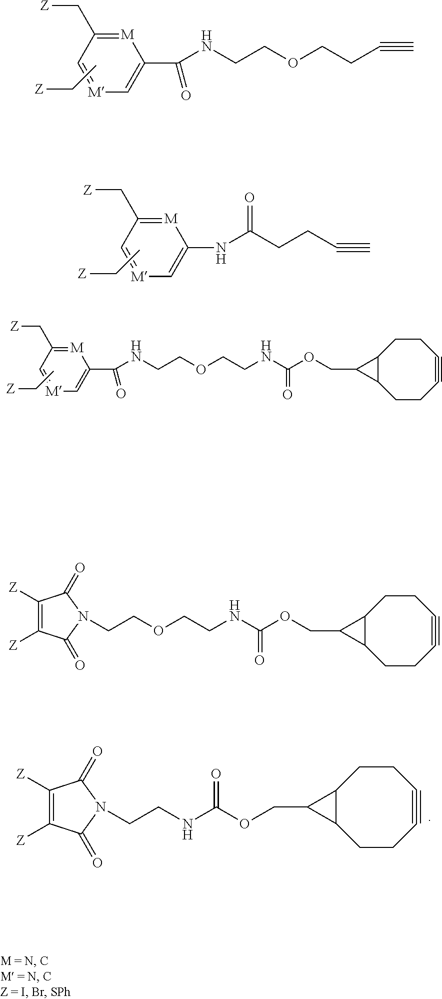

Preferably, the reduction of antibody A to form half-antibody A and antibody B to form half-antibody B is conducted in a reducing agent such as L-cysteine, dithiothreitol, beta-mercapto ethanol, cysteamine, TCEP (tris(2-carboxyethyl)phosphine), 2-MEA (2-Mercaptoethylamine), and combinations thereof.

Preferably, antibodies A and B are monoclonal antibodies. Monoclonal antibodies may be produced by hybridoma methods or by recombinant DNA and protein expression methods. Further, antibodies A and B are full-length antibodies or are antibody fragments.

The antibodies A and B have a CPPC core hinge region sequence or a CPSC core hinge region sequence or a SPPC core hinge region sequence or a SPSC core hinge region sequence (EU-index numbering: residues 226-229; Kabat numbering: residues 239-242). Further, step (d) incubating further comprises the step of adding a reducing agent, wherein the reducing gent is selected from the group consisting of L-cysteine, dithiothreitol, beta-mercapto ethanol, cysteamine, TCEP (tris(2-carboxyethyl)phosphine), 2-MEA (2-Mercaptoethylamine), and combinations thereof.

The quality and purity of the resulting bispecific antibodies can be analyzed using routine biochemical techniques, such as absorbance measurements, HP-SEC, SDS-PAGE, native PAGE, and RP-HPLC. It should be noted that the disclosed method generally avoids any purification step because of the specificity of the affinity the linker of formula I for the linker of formula II. However, there are various purification steps provided in US2010/0105874, the disclosure of which is incorporated by reference herein.

The disclosed process further comprises the step of formulating the bispecific antibody for therapeutic use. This is accomplished by a formulation of an effective amount of a bispecific antibody in an aqueous solution that is suitable for human use, in particular suitable for parenteral or intravenous administration.

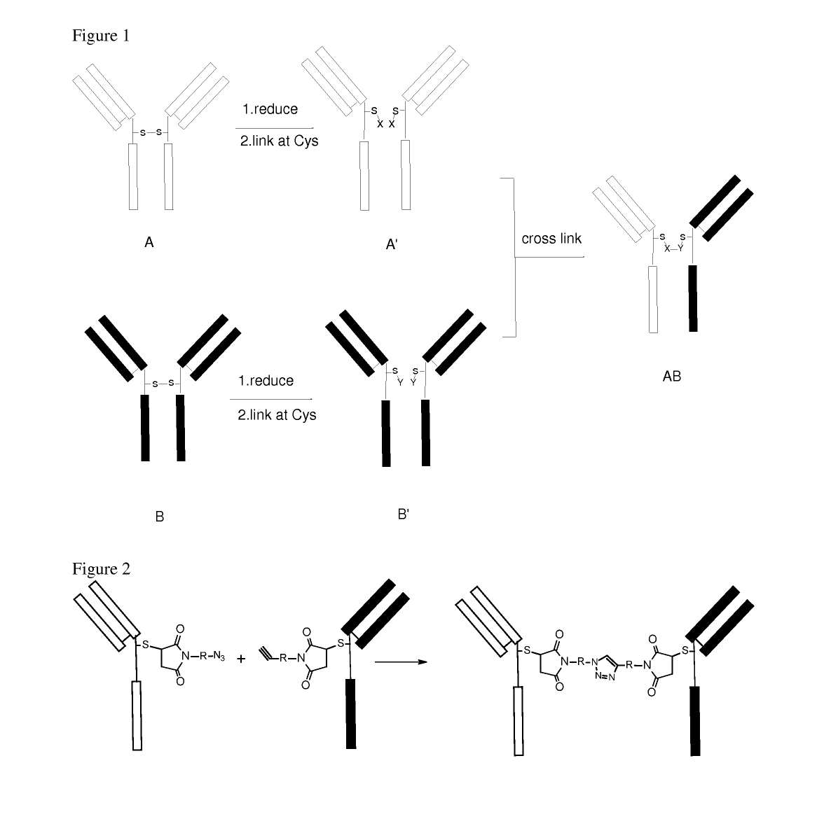

FIG. 2 shows a scheme to generate bispecific monoclonal antibody (mAb) via chemical conjugation. A bispecific mAb described herein is made up of two half-antibody fragments chemically linked at the hinge region. The process of bispecific mAb generation involves three main steps (FIG. 2). The first step is a selective reduction of hinge disulfides in two different mAb A and B respectively. The second step is an induction of intrachain-link between two cysteines on the same heavy chain in each mAb through a linker X or Y. The intrachain-link process produces two chemically locked mAb fragments A′ and B′. In the last step, two mAb fragments are linked together through a chemical ligation between X and Y to form a bispecific antibody AB.

IgG1 with hinge mutations (CPSC) and wt IgG4 are used in this study.

The first step is to reduce each of antibody A and antibody B. In one embodiment, the antibody (10 mg) was treated with 10 molar equivalents of 2-mercaptoethyl-amine (2-MEA) in 0.1M PBS pH 7.4, 1.0 mM diethylenetriaminepentaacetic acid (DTPA) for 2 h at 37° C. Excess 2-MEA was purified away from the partially reduced mAb using 50 kDa filter centrifuge tubes with centrifugation conducted at 3,000 RPM for 20 minutes. A total of three washes with 0.1M PBS were conducted. The protein concentration was quantified using an absorbance value of 1.58 at 280 nm for a 1.0 mg/mL solution, and the molar concentration determined using a molecular weight of 150,000 g/mol.

In another embodiment of the reduction step, the antibody (10 mg) was treated with 3.0 molar equivalents of dithiothreitol (DTT) in 0.1M PBS pH 7.4, 1.0 mM diethylenetriaminepentaacetic acid (DTPA) for 2 h at 24° C. The excess DTT was purified away from the partially reduced mAb using 50 kDa filter centrifuge tubes with centrifugation conducted at 3,000 RPM for 20 minutes. A total of 3 washes with 0.1M PBS were conducted.

In another embodiment of the reduction step, the mAb (10 mg) was treated with 2.0 molar equivalents of tris (2-carboxyethyl)-phosphine (TCEP) in 0.1M PBS pH 8.0, 1.0 mM diethylenetriaminepentaacetic acid (DTPA) for 2 h at 24° C. The mAb concentration was 8.0 mM. Without purification, the partially reduced mAb was used in conjugation step directly.

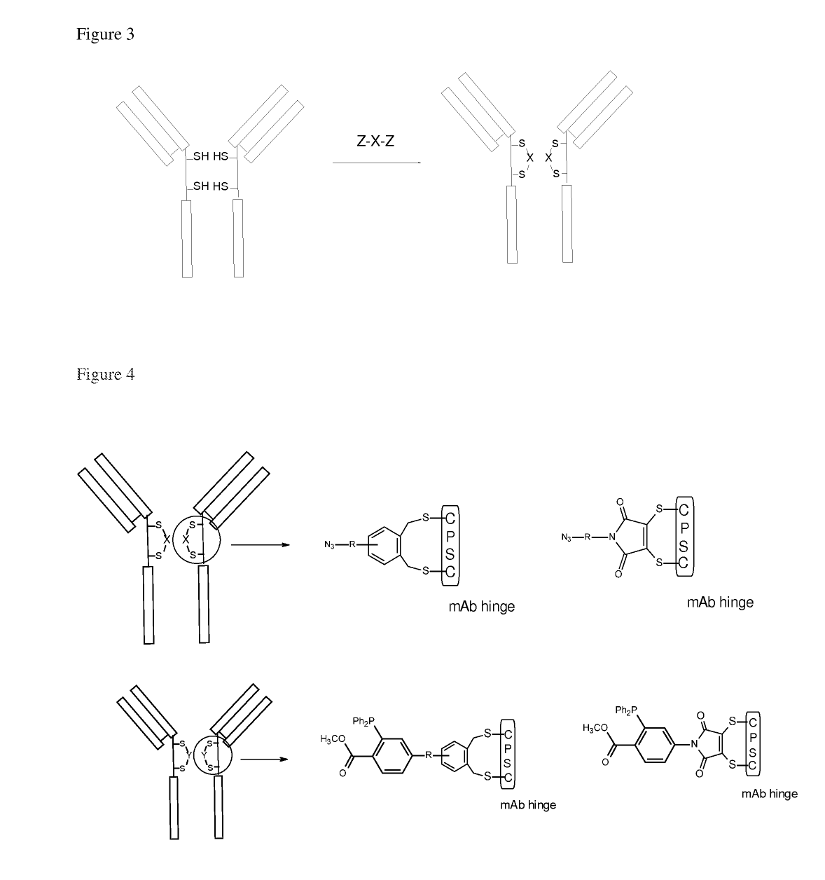

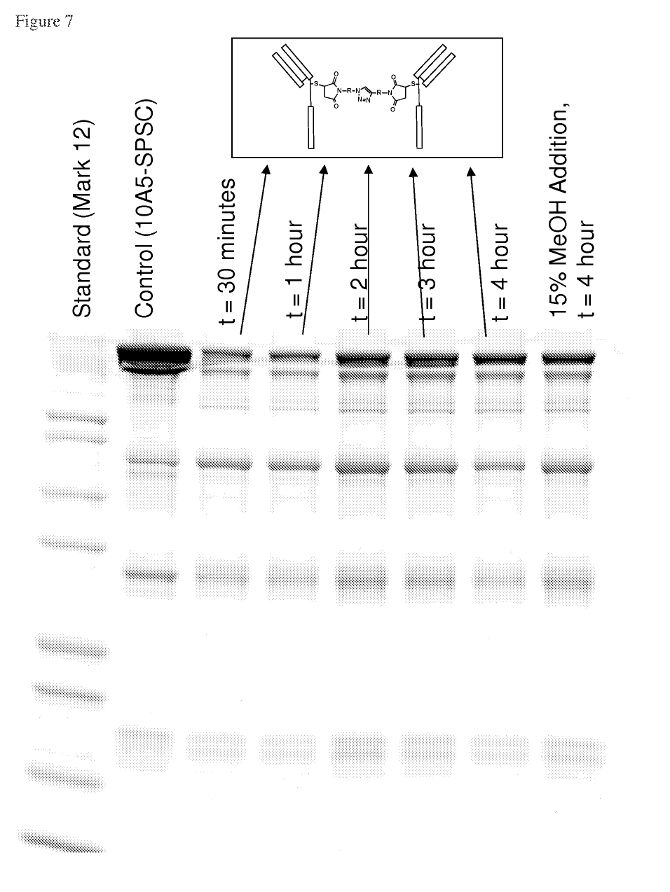

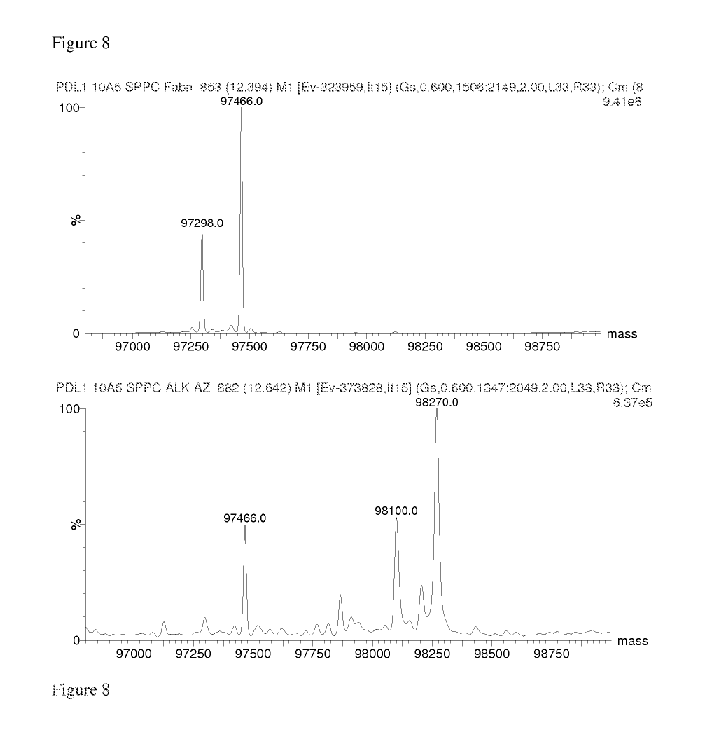

The second step is the conjugation step. A partially reduced mAb “Antibody A” from a reduction step in 0.1M PBS was added to 2.5 molar equivalents of cross linking agent Z—X—Z (FIG. 2 and FIG. 3). The cross linking agent was taken from a pre-prepared stock solution in DMSO (1 mg/mL). In the reaction mixture, partially reduced antibody concentration was 8.0 mg/mL and DMSO content was 5% (v/v). The conjugation was carried out for 2 hr at 24° C. Cysteine (1 mM final) was used to quench any unreacted, excess cross linking agents. Conjugated mAb was purified using PD-10 columns equilibrated with phosphate buffered saline. The conjugated mAb structures are illustrated in FIG. 4. Under the same conditions, second mAb (Antibody B) was conjugated with crossing linking agent Z—Y—Z (FIG. 5 and FIG. 6) and purified. The conjugated mAb structures are illustrated in FIG. 7 and FIG. 8.

The third step is the inter-chain conjugation step. The click conjugation for interchain cross-link is illustrated in FIG. 9. In brief, to azide-decorated antibody fragments (3.0 mg) in 0.5 mL of PBS (0.1M, pH 7.4) is added 3.0 mg of alkyne-decorated antibody fragments in 0.5 mL of PBS (0.1M, pH 7.4). To this mixture is added 50 μL of acetonitrile and the final content of acetonitrile is 5% (v/v). After 3 hr of reaction at room temperature, the mixture is purified using 100 kDa filter centrifuge tubes with centrifugation conducted at 3,000 RPM for 20 minutes. The mixture is washed with PBS for 3 times and the resulted product is subject to in vitro characterization.

### Example 1

This example shows the synthesis of a bispecific antibody according to the disclosed process. FIG. 4 shows a scheme to generate bispecific monoclonal antibody (mAb) by chemical conjugation to two Cys residues in the hinge region of an IgG class antibody. The disclosed bispecific mAbs are made up of two half-antibody fragments chemically linked at their respective hinge regions. The process to synthesize bispecific mAbs involves three main steps shown in FIG. 5. The first step is a selective reduction of hinge disulfides in two different mAb's, A and B respectively. The second step is an induction of intrachain-link between two cysteines on the same heavy chain in each mAb through a linker X or Y. The intrachain-link process produces two chemically locked mAb fragments A′ and B′. In the last step, two mAb fragments are linked together through a chemical ligation between X and Y to form a bispecific antibody AB.

More specifically, we obtained antibody “A” an IgG1 with hinge mutations (CPSC) and antibody “B” a wild type IgG4. The first step was antibody reduction. Condition 1: The antibodies (10 mg) were separately treated with 10 molar equivalents of 2-mercaptoethyl-amine (2-MEA) in 0.1M PBS pH 7.4, 1.0 mM diethylenetriaminepentaacetic acid (DTPA) for 2 h at 37° C. Excess 2-MEA was purified away from the partially reduced mAb using 50 kDa filter centrifuge tubes with centrifugation conducted at 3,000 RPM for 20 minutes. A total of three washes with 0.1M PBS were conducted. The protein concentration was quantified using an absorbance value of 1.58 at 280 nm for a 1.0 mg/mL solution, and the molar concentration determined using a molecular weight of 150,000 g/mol.

Condition 2: The antibody (10 mg) was treated with 3.0 molar equivalents of dithiothreitol (DTT) in 0.1M PBS pH 7.4, 1.0 mM diethylenetriaminepentaacetic acid (DTPA) for 2 h at 24° C. The excess DTT was purified away from the partially reduced mAb using 50 kDa filter centrifuge tubes with centrifugation conducted at 3,000 RPM for 20 minutes. A total of 3 washes with 0.1M PBS were conducted.

Condition 3: The mAb (10 mg) was treated with 2.0 molar equivalents of tris (2-carboxyethyl)-phosphine (TCEP) in 0.1M PBS pH 8.0, 1.0 mM diethylenetriaminepentaacetic acid (DTPA) for 2 h at 24° C. The mAb concentration was 8.0 mM. Without purification, the partially reduced mAb was used in conjugation directly.

### Example 2

This example shows that the bispecific antibody made in Example 1 retained both of its original half Mab binding characteristics.

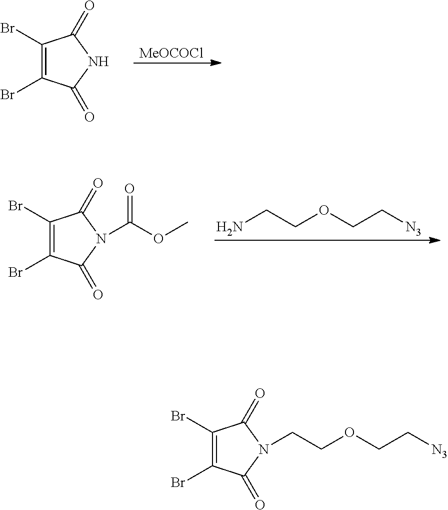 [SMILES: "MeOCOCl" ; O=C1NC(=O)C(Br)=C1Br ; COC(=O)N1C(=O)C(Br)=C(Br)C1=O ; [N-]=[N+]=NCCOCCN ; [N-]=[N+]=NCCOCCN1C(=O)C(Br)=C(Br)C1=O]

### Synthesis of 1-(2-(2-azidoethoxy)ethyl)-3,4-dibromo-1H-pyrrole-2,5-dione

To 2.5 g of 3,4-dibromo-1H-pyrrole-2,5-dione (10 mmol) and 1 g of NMM in 60 mL of THF, MeOCOCl (10 mmol, 940 mg in 10 ml DCM) was added dropwise, stirred for 20 min, then the reaction solution was diluted with 6o mL of DCM, washed 3 time by water, the organic phase was stirred by sodium sulfate anhydrous, concentrated, 2.65 g of methyl 3,4-dibromo-2,5-dioxo-2H-pyrrole-1(5H)-carboxylate was obtained. To 311 mg, 1 mmol of this compound, 2-(2-azidoethoxy)ethanamine (130 mg, 1 mmol) and 5 mL DCM was added, TLC shown the reaction finished in 20 min, then extracted by DCM and brine, washed by NH₄Cl solution, dried on sodium sulfate anhydrous, and then concentrated for column purification, flashed by 2:1 hexane and ethyl ethylate, 230 mg of 1-(2-(2-azidoethoxy)ethyl)-3,4-dibromo-1H-pyrrole-2,5-dione obtained. ¹HNMR: 3.32 ppm (t, J=5.0 Hz, 1H), 3.40 ppm (t, J=5.0 Hz, 1H), 3.50 ppm (q, J=5.0 Hz, 1H), 3.62 ppm (t, J=5.0 Hz, 1H), 3.63-3.69 ppm (m, 3H), 3.84 ppm (t, J=5 hz, 1H). Fw: 365.9, C₈H₈Br₂N₄O₃; Mass Peaks (1:2:1): 366.9, 368.9, 370.9.

### Example 3

This example illustrates chemical generation of a bispecific antibody using a single Cys residue located in the hinge region of an IgG class antibody. The starting mAbs described herein contain an engineered hinge region where one Cys at the same position on each chain was mutated to Ser, thus resulting in a hinge with only a single disulfide left. The process of bispecific mAb generation involves three main steps (FIG. 1). The first step is a selective reduction of hinge disulfide in two different mAb A and B respectively. The second step is an induction of a functional moiety X or Y via a cysteine-based conjugation. The Cys-link step produces two chemically locked mAb fragments A′ and B′. In the last step, two mAb fragments are linked together through a chemical ligation between X and Y to form a bispecific antibody AB. An IgG1 monoclonal antibody with a hinge region mutation (SPPC) were used in this study.

Condition 1: The antibody (10 mg) was treated with 10 molar equivalents of 2-mercaptoethyl-amine (2-MEA) in 0.1M PBS pH 7.4, 1.0 mM diethylenetriaminepentaacetic acid (DTPA) for 2 h at 37° C. Excess 2-MEA was purified away from the partially reduced mAb using 50 kDa filter centrifuge tubes with centrifugation conducted at 3,000 RPM for 20 minutes. A total of three washes with 0.1M PBS were conducted. The protein concentration was quantified using an absorbance value of 1.58 at 280 nm for a 1.0 mg/mL solution, and the molar concentration determined using a molecular weight of 150,000 g/mol.

Condition 2: The antibody (10 mg) was treated with 3.0 molar equivalents of dithiothreitol (DTT) in 0.1M PBS pH 7.4, 1.0 mM diethylenetriaminepentaacetic acid (DTPA) for 2 h at 24° C. The excess DTT was purified away from the partially reduced mAb using 50 kDa filter centrifuge tubes with centrifugation conducted at 3,000 RPM for 20 minutes. A total of 3 washes with 0.1M PBS were conducted.

Condition 3: The mAb (10 mg) was treated with 2.0 molar equivalents of tris (2-carboxyethyl)-phosphine (TCEP) in 0.1M PBS pH 8.0, 1.0 mM diethylenetriaminepentaacetic acid (DTPA) for 2 h at 24° C. The mAb concentration was 8.0 mM. Without purification, the partially reduced mAb was used in conjugation directly.

We claim:

1. A process for making a bi-specific antibody “AB” or “BA” from a first antibody “A” and a second antibody “B” comprising:
(a) contacting said first antibody A with a reducing agent under conditions sufficient to cleave substantially all disulfide linkages between the heavy chains in the hinge region to yield a pair of first antibody fragments A′, each comprising a single light chain attached to a single heavy chain, wherein the heavy chain has one or more reactive thiol groups formed from a reduction of said disulfide linkages;
(b) attaching a first hetero-bi-functional linker to said first antibody fragment A′, said first hetero-bi-functional linker comprising (i) a first thiol-reactive functional group for covalent attachment to a reactive thiol group of said heavy chain of said first antibody fragment, and (ii) an azide, to thereby form an azide-functionalized first antibody fragment;
(c) contacting said second antibody B with a reducing agent under conditions sufficient to cleave substantially all disulfide linkages between the heavy chains in the hinge region, to yield a pair of second antibody fragments B′, each comprising a single light chain attached to a single heavy chain, wherein the heavy chain has one or more reactive thiol groups formed from the reduction of said disulfide linkages;
(d) attaching a second hetero-bi-functional linker to said second antibody fragment B′, said second hetero-bi-functional linker comprising: (i) a second thiol-reactive functional group for covalent attachment to a reactive thiol group of said heaving chain of said second antibody fragment, and (ii) an alkyne; to thereby form an alkyne-functionalized second antibody fragment; and
(e) reacting said azide functionalized first antibody fragment with said alkyne functionalized second antibody fragment to covalently attach said first antibody fragment to said second antibody fragment via cyloaddition of said azide to said alkyne, to form a chemically-locked bi-specific antibody “AB” or “BA.”

2. The process for making a bi-specific antibody “AB” or “BA” from a first antibody “A” and a second antibody “B” according to claim 1, wherein said first hetero-bi-functional linker has the form Q-L-N₃, wherein Q is a thiol-reactive functional group comprising an alkyl halide, benzyl halide, maleimide, halo-maleamide, or dihalo-maleimide; and L is a hydrocarbon linker having from 3-60 atoms, and N₃ is an azide group.

3. The process for making a bi-specific antibody “AB” or “BA” from a first antibody “A” and a second antibody “B” according to claim 1, wherein said first hetero-bi-functional linker has the form Q-L-N₃, wherein Q is a thiol-reactive functional group comprising a maleimide, halo-maleamide, or dihalo-maleimide group; and L is a hydrocarbon linker having from 3-60 atoms in a polymer configuration having units —(CH₂CH₂—O)ₙ— and/or —(O—CH₂CH₂)ₙ—, wherein “n” is independently an integer from 1-20; and N₃ is an azide group.

4. The process for making a bi-specific antibody “AB” or “BA” from a first antibody “A” and a second antibody “B” according to claim 2, wherein said first hetero-biofunctional linker has the form: 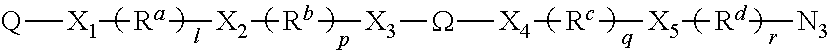
wherein,
Q is a thiol-reactive group of the form: 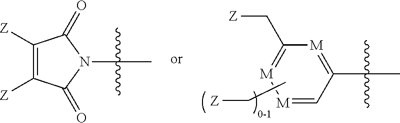
wherein Z is independently selected from the group consisting of H, Br, I, and SPh, with the proviso that at least one occurrence of Z is not H; and M is independently either CR* or N;
wherein X₁, X₂, X₃, X₂, X₄ and X₅ are independently selected from the group consisting of a bond, —O—, —NR^{N}—, —N═C—, —C═N—, —N═N—, —CR*═CR*— (cis or trans), —C≡C—, —(C═O)—, —(C═O)—O—, —(C═O)—NR^{N}—, —(C═O)—(CH₂)ₙ—, —(C═O)—O—(CH₂)ₙ—, —(C═O)—NR^{N}—(CH₂)ₙ—, and —(C═O)—NR^{N}—(CH₂CH₂—O)ₙ—, wherein “n” is either zero or an integer from 1-10;
wherein R^{a}, R^{b}, R^{c}, and R^{d} are independently selected from the group consisting of —O—, —NR^{N}—, —CH₂—, —(CH₂)ₙ—, —(CR*₂)ₙ—, —(CH₂CH₂—O)ₙ—, —(CR*₂CR*₂—O)ₙ—, —(O—CH₂CH₂)ₙ—, —(O—CR*₂CR*₂)ₙ—, —CR*═CR*— (cis or trans), —N═C—, —C═N—, —N═N—, —C≡C—, —(C═O)—, —(CH₂)ₙ—(C═O)—, —(C═O)—(CH₂)ₙ—, —(CH₂)ₙ—(C═O)—(CH₂)ₙ—, —O—(C═O)—, —(C═O)—O—, —O—(C═O)—O—, —(CH₂)ₙ—(C═O)—O—, —O—(C═O)—(CH₂)ₙ, —(C═O)—O—(CH₂)ₙ—, —(CH₂)ₙ—O—(C═O)—, —(CH₂)ₙ—(C═O)—O—(CH₂)ₙ—, —(CH₂)ₙ—O—(C═O)—(CH₂)ₙ—, —NR^{N}—(C═O)—, —(C═O)—NR^{N}—, —NR^{N}—(C═O)—O—, —O—(C═O)—NR^{N}—, —NR^{N}—(C═O)—NR^{N}—, —(CH₂)ₙ—(C═O)—NR^{N}—, —NR^{N}—(C═O)—(CH₂)ₙ, —(C═O)—NR^{N}—(CH₂)ₙ—, —(CH₂)ₙ—NR^{N}—(C═O)—, —(CH₂)ₙ—(C═O)—NR^{N}—(CH₂)ₙ—, —(CH₂)ₙ—NR^{N}—(C═O)—(CH₂)ₙ—, —(C═O)—NR^{N}—(CH₂CH₂—O)ₙ—, —(CH₂CH₂—O)ₙ—(C═O)—NR^{N}—, —(CH₂)ₙ—(C═O)—NR^{N}—(CH₂CH₂—O)ₙ—, —(CH₂CH₂—O)ₙ—(C═O)—NR^{N}—(CH₂)ₙ—, or a 2-8 membered cyclic hydrocarbon, heterocycle, aryl, or heteroaryl ring; wherein “n” is, independently either zero or an integer from 1-10; and wherein “l”, “p”, “q”, and “r” are independently either zero or integers from 1-10;
Ω is either a bond or is a C₃₋₂₆ hydrocarbon ring or fused ring system, optionally comprising up to four fused rings, wherein each ring has from 3-8 members and optionally comprising from 1-4 heteroatoms selected from O, S, and N in each ring;
wherein R* and R^{N} are independently either H or a C₁₋₁₂ hydrocarbon, optionally substituted with 1-6 heteroatoms selected from the group consisting of a halogen, O, S, and N; and
wherein R* and/or R^{N} may together from a 3-8 membered ring.

5. The process for making a bi-specific antibody “AB” or “BA” from a first antibody “A” and a second antibody “B” according to claim 4, wherein Q is maleimide, bromo-maleimide, or dibromomaleimide.

6. The process for making a bi-specific antibody “AB” or “BA” from a first antibody “A” and a second antibody “B” according to claim 1, wherein said second hetero-bi-functional linker has the form Q-L-G, wherein Q is a thiol-reactive functional group comprising an alkyl halide, benzyl halide, maleimide, halo-maleamide, or dihalo-maleimide; and L is a hydrocarbon linker having from 3-60 atoms, and G is an alkyne containing group.

7. The process for making a bi-specific antibody “AB” or “BA” from a first antibody “A” and a second antibody “B” according to claim 1, wherein G is —C≡CH.

8. The process for making a bi-specific antibody “AB” or “BA” from a first antibody “A” and a second antibody “B” according to claim 1, wherein G comprises a C8 ring having a —C≡C— bond.

9. The process for making a bi-specific antibody “AB” or “BA” from a first antibody “A” and a second antibody “B” according to claim 8, wherein G has the form:

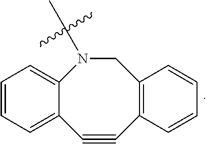 [SMILES: *N1Cc2ccccc2C#Cc2ccccc21]

10. The process for making a bi-specific antibody “AB” or “BA” from a first antibody “A” and a second antibody “B” according to claim 8, wherein G has the form:

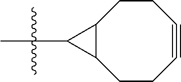 [SMILES: *C1C2CCC#CCCC12]

11. The process for making a bi-specific antibody “AB” or “BA” from a first antibody “A” and a second antibody “B” according to claim 1, wherein said second hetero-bi-functional linker has the form Q-L-G, wherein Q is a thiol-reactive functional group comprising a maleimide, halo-maleamide, or dihalo-maleimide group; and L is a hydrocarbon linker having from 3-60 atoms and comprising a polymer having units —(CH₂CH₂—O)ₙ— or —(O—CH₂CH₂)ₙ—, wherein “n” is independently an integer from 1-20; and G is a C₈₋₂₀ hydrocarbon comprising a C8 ring having a —C≡C— bond capable of undergoing a 1,3 dipolar cycloaddition reaction with an azide.

12. The process for making a bi-specific antibody “AB” or “BA” from a first antibody “A” and a second antibody “B” according to claim 10, wherein said second hetero-biofunctional linker has the form: 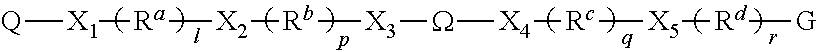
wherein,
Q is a thiol-reactive group of the form: 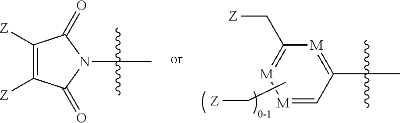
wherein Z is independently selected from the group consisting of H, Br, I, and SPh, with the proviso that at least one occurrence of Z is not H; and M is independently either CR* or N;
G is a C₈₋₂₀ hydrocarbon group comprising a C₈ ring having a —C≡C— bond capable of undergoing a 1,3 dipolar cycloaddition reaction with said azide;
X₁, X₂, X₃, X₂, X₄ and X₅ are independently selected, at each occurrence, from the group consisting of a bond, —O—, —NR^{N}—, —N═C—, —C═N—, —N═N—, —CR*═CR*— (cis or trans), —C≡C—, —(C═O)—, —(C═O)—O—, —(C═O)—NR^{N}—, —NR^{N}—(C═O)—, —NR^{N}—(C═O)—O—, —(C═O)—(CH₂)ₙ—, —(C═O)—O—(CH₂)ₙ—, —(C═O)—NR^{N}—(CH₂)ₙ—, and —(C═O)—NR^{N}—(CH₂CH₂—O)ₙ—, wherein “n” is either zero or an integer from 1-10;
R^{a}, R^{b}, R^{c}, and R^{d} are independently selected from the group consisting of —O—, —NR^{N}—, —CH₂—, —(CH₂)ₙ—, —(CR*₂)ₙ—, —(CH₂CH₂—O)ₙ—, —(CR*₂CR*₂—O)ₙ—, —(O—CH₂CH₂)ₙ—, —(O—CR*₂CR*₂)ₙ—, —CR*═CR*— (cis or trans), —N═C—, —C═N—, —N═N—, —C≡C—, —(C═O)—, —(CH₂)ₙ—(C═O)—, —(C═O)—(CH₂)ₙ—, —(CH₂)ₙ—(C═O)—(CH₂)ₙ—, —O—(C═O)—, —(C═O)—O—, —O—(C═O)—O—, —(CH₂)ₙ—(C═O)—O—, —O—(C═O)—(CH₂)ₙ, —(C═O)—O—(CH₂)ₙ—, —(CH₂)ₙ—O—(C═O)—, —(CH₂)ₙ—(C═O)—O—(CH₂)ₙ—, —(CH₂)ₙ—O—(C═O)—(CH₂)ₙ—, —NR^{N}—(C═O)—, —(C═O)—NR^{N}—, —NR^{N}—(C═O)—O—, —O—(C═O)—NR^{N}—, —NR^{N}—(C═O)—NR^{N}—, —(CH₂)ₙ—(C═O)—NR^{N}—, —NR^{N}—(C═O)—(CH₂)ₙ, —(C═O)—NR^{N}—(CH₂)ₙ—, —(CH₂)ₙ—NR^{N}—(C═O)—, —(CH₂)ₙ—(C═O)—NR^{N}(CH₂)ₙ—, —(CH₂)ₙ—NR^{N}—(C═O)—(CH₂)ₙ—, —(C═O)—NR^{N}—(CH₂CH₂—O)ₙ—, —(CH₂CH₂—O)ₙ—(C═O)—NR^{N}—, —(CH₂)ₙ—(C═O)—NR^{N}—(CH₂CH₂—O)ₙ—, —(CH₂CH₂—O)ₙ—(C═O)—NR^{N}—(CH₂)ₙ—, or a 2-8 membered cyclic hydrocarbon, heterocycle, aryl, or heteroaryl ring; wherein “n” is, independently either zero or an integer from 1-10; and wherein “l”, “p”, “q”, and “r” are independently either zero or an integer from 1-10;
Ω is independently a bond or is a C₃₋₂₆ hydrocarbon ring or fused ring system, optionally comprising up to four fused rings, each ring having from 3-8 members and optionally comprising from 1-4 heteroatoms independently selected from O, S, and N in each ring;
wherein R* and R^{N} are, independently at each occurrence, either H or a C₁₋₁₂ hydrocarbon, optionally substituted with 1-6 heteroatoms selected from halogen, O, S, and N; and wherein an two groups R* and/or R^{N} may together from a 3-8 membered ring.

13. The process for making a bi-specific antibody “AB” or “BA” from a first antibody “A” and a second antibody “B” according to claim 1, wherein said cycloaddition reaction occurs in the presence of copper ions.

14. The process for making a bi-specific antibody “AB” or “BA” from a first antibody “A” and a second antibody “B” according to claim 1, wherein said cycloaddition reaction occurs at neutral pH.

15. The process for making a bi-specific antibody “AB” or “BA” from a first antibody “A” and a second antibody “B” according to claim 1, wherein at least 90% of the disulfide linkages between the heavy chains and light chains remain substantially intact following cleavage of the disulfide bonds in the hinge region.

16. The process for making a bi-specific antibody “AB” or “BA” from a first antibody “A” and a second antibody “B” according to claim 1, wherein antibody A or antibody B are IgG1 immunoglobulins.

17. The process for making a bi-specific antibody “AB” or “BA” from a first antibody “A” and a second antibody “B” according to claim 1, wherein antibody A or antibody B are IgG4 immunoglobulins.

18. A process for generation of a chemically-locked bispecific antibody “AB” or “BA” from IgG1, IgG2 or IgG4 class antibody or Fab2 fragment thereof “A” and IgG1, IgG2 or IgG4 class antibody or fragment thereof “B” comprising:
(a) reducing a first antibody “A” having a hinge residue sequence (EU-index numbering: residues 226-229) selected from the group consisting of CPPC, CPSC, SPPC, and SPSC and antibody “B” having a hinge residue sequence (EU-index numbering: residues 226-229) selected from the group consisting of CPPC, CPSC, SPPC, and SPSC; to form half-antibody A and half-antibody-B, wherein antibody A binds to a first target and antibody B binds to a second target, whereby the reducing conditions break any inter-chain or intra-chain disulfide bonds in the hinge region of antibody A and antibody B;
(b) linking a first compound to one or both Cys residues 226 or/and 229 (EU-index numbering: residues 226 or/and 229) of the antibody hinge core sequence of half-antibody A to form a linked half-antibody A wherein the first compound has a structure selected from the group consisting of: 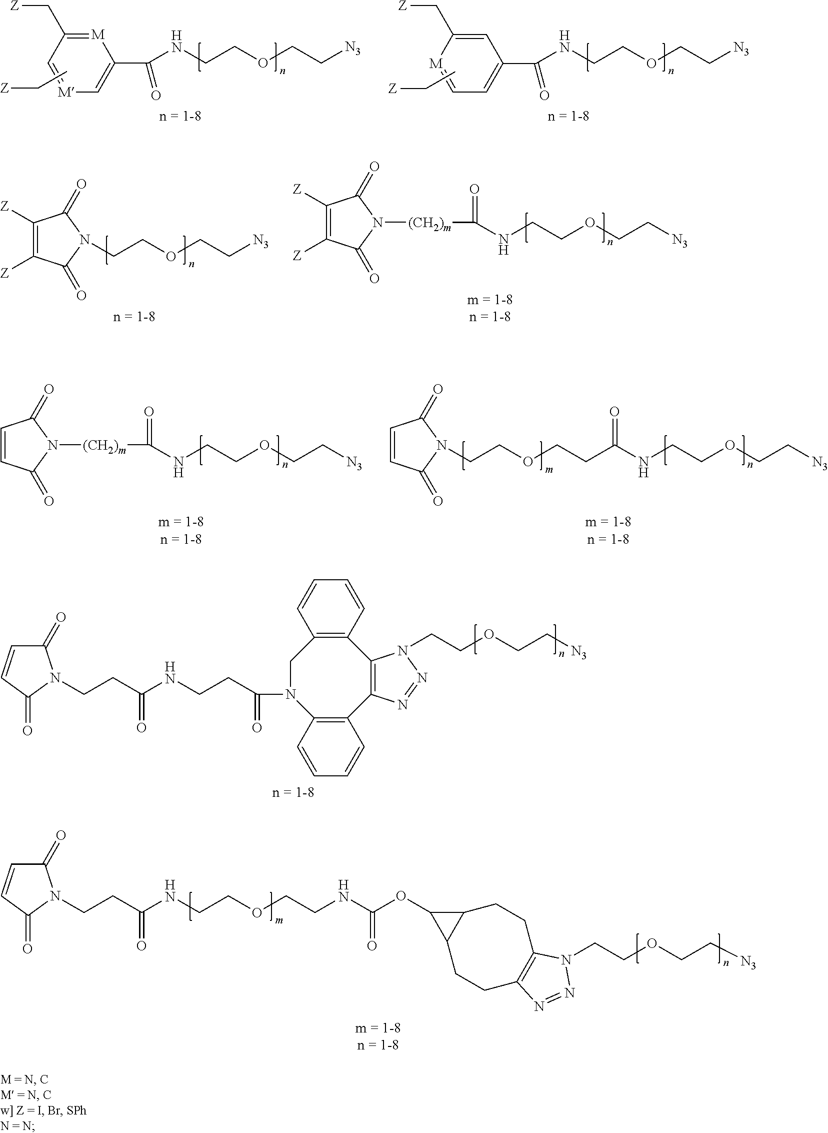
(c) linking a second compound to one or both Cys residues 226 and 229 of hinge core sequence of antibody B with the hinge residue sequence (residues 226-229) CPPC or CPSC or SPPC or SPSC to form a linked antibody B wherein the second compound has a structure selected from the group consisting of: 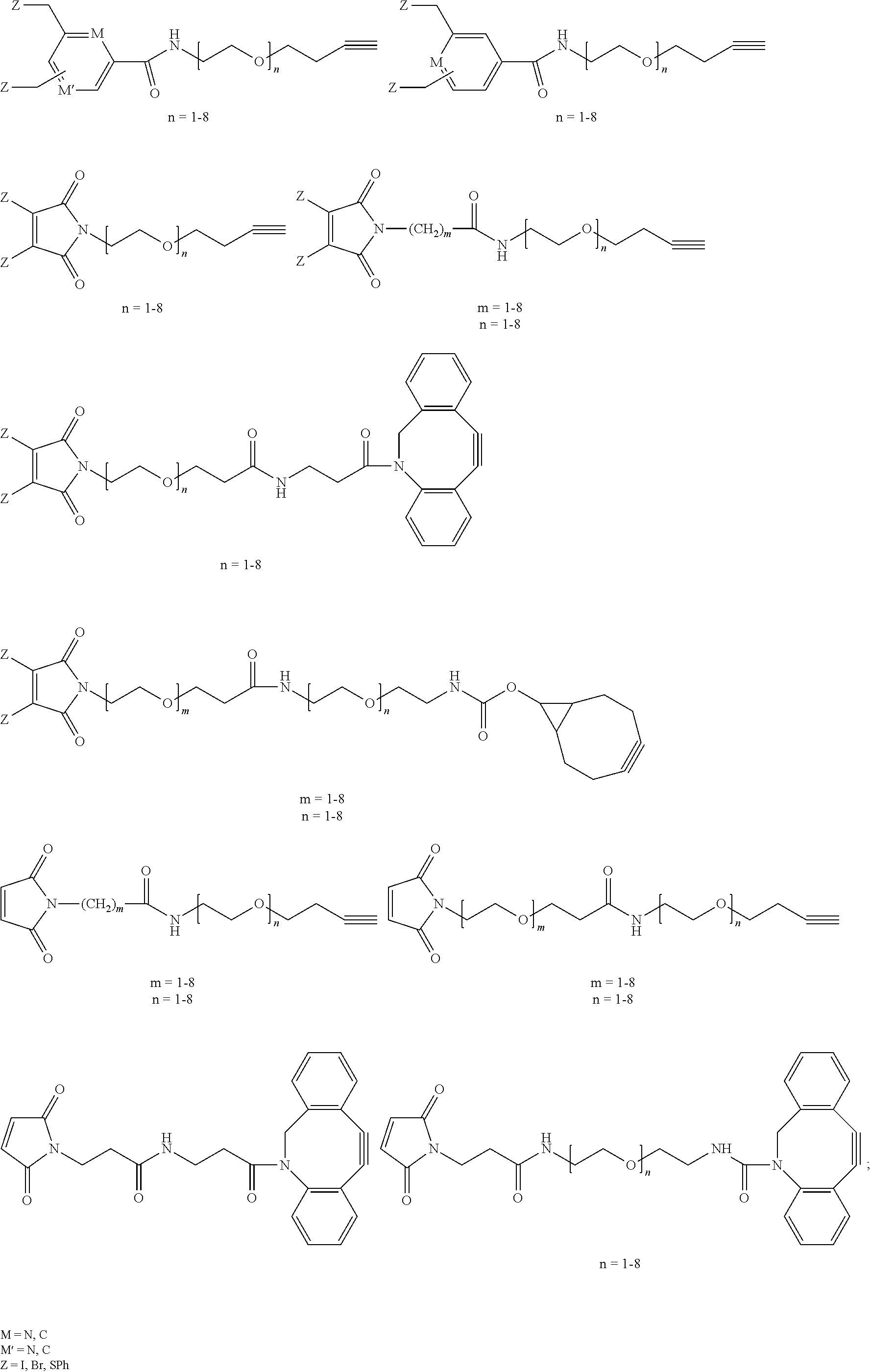
(d) incubating approximately equal molar amounts of linked antibody A with linked antibody B under neural conditions to form the chemically-locked bispecific antibody AB.

19. The process for generation of a chemically-locked bispecific antibody of claim 18, wherein the reduction of antibody A to form half-antibody A and the reduction of antibody B to form half-antibody B is conducted in a reducing agent, wherein the reducing agent is selected from the group consisting of L-cysteine, dithiothreitol, beta-mercapto ethanol, cysteamine, TCEP (tris(2-carboxyethyl)phosphine), 2-MEA (2-Mercaptoethylamine), and combinations thereof.

20. The process for generation of a chemically-locked bispecific antibody of claim 18, wherein the hinge region of antibody A, having two Cys residues (EU-index numbering: residues 226 or/and 229), is linked with a moiety A having the structure selected from the group consisting of: 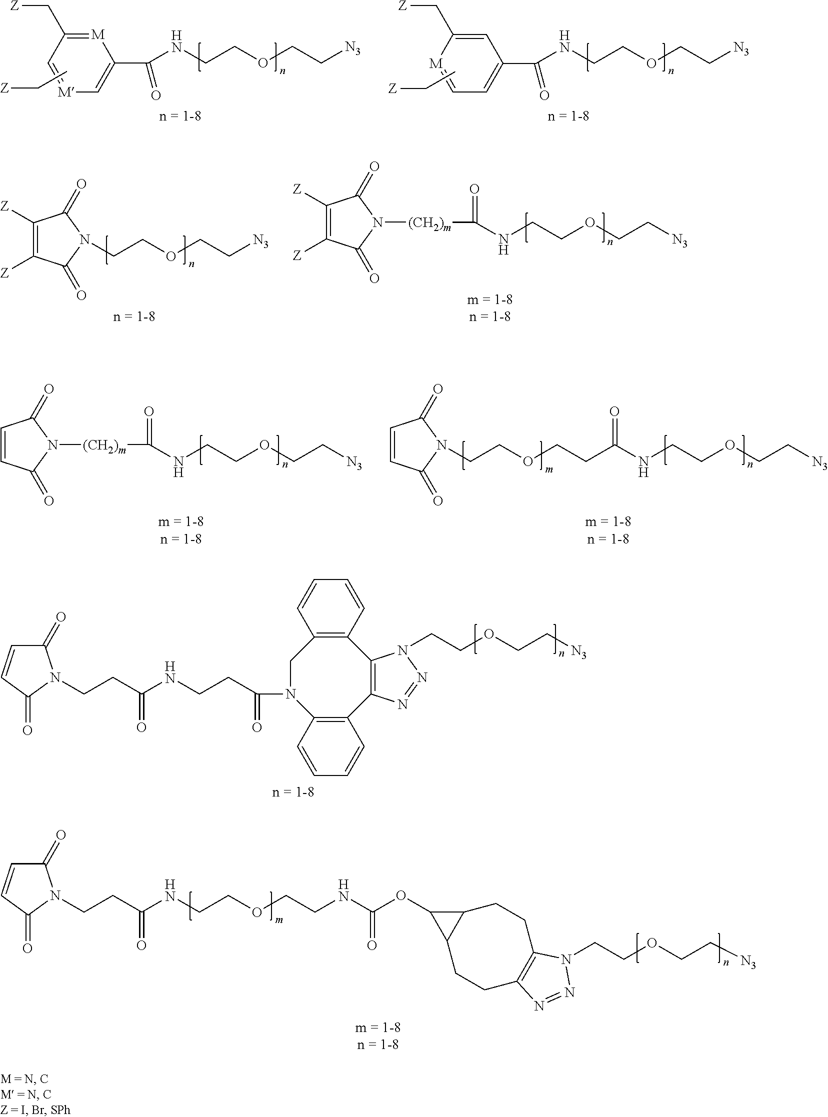
wherein N₃ is —N═N═N.

21. The process for generation of a chemically-locked bispecific antibody of claim 18, wherein the hinge region of antibody B, having one or two Cys residues (EU-index numbering: residues 226 or/and 229), is linked with a moiety B having the structure selected from the group consisting of: 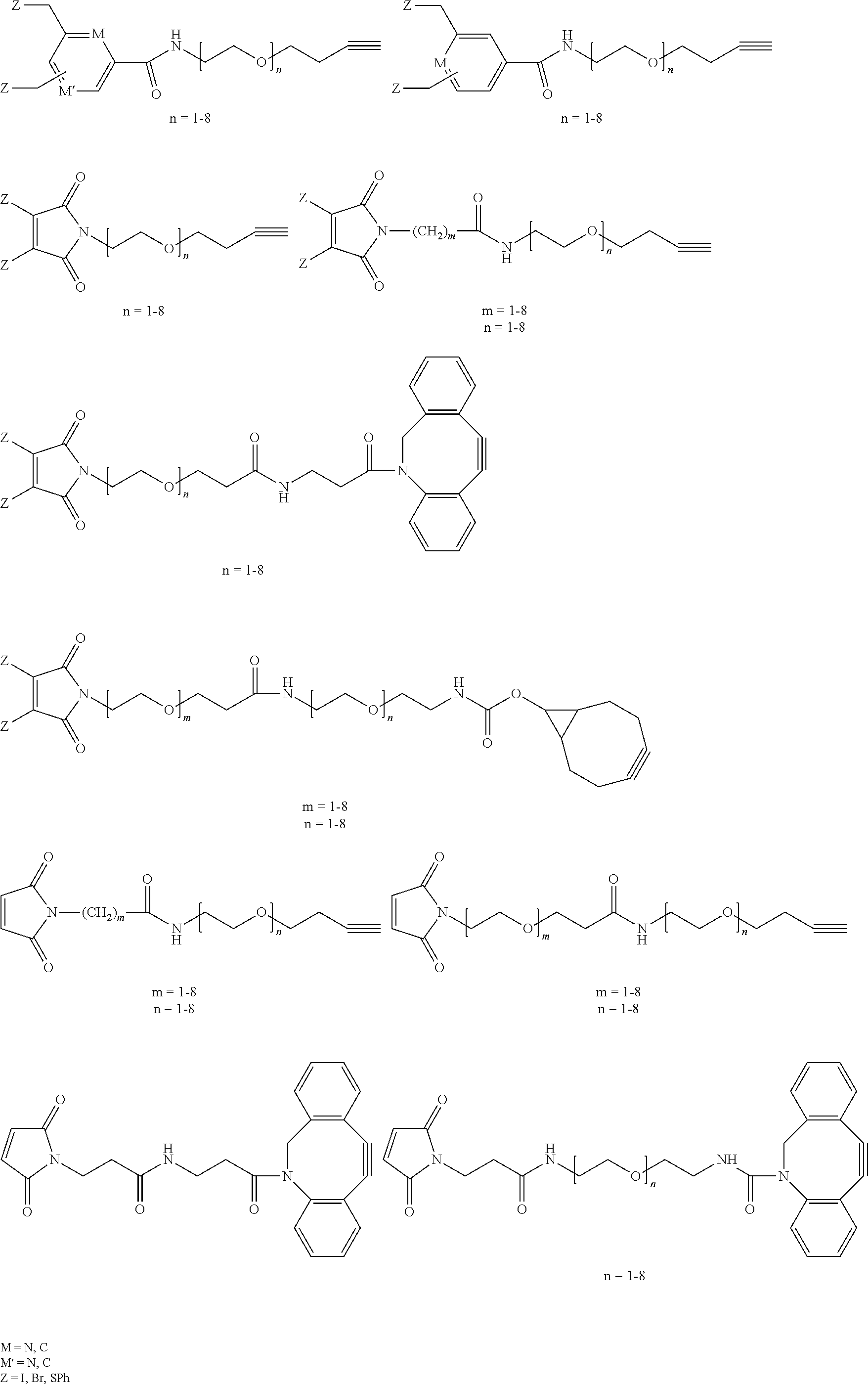
to form a linked half-antibody A having a structure selected from the group consisting of: 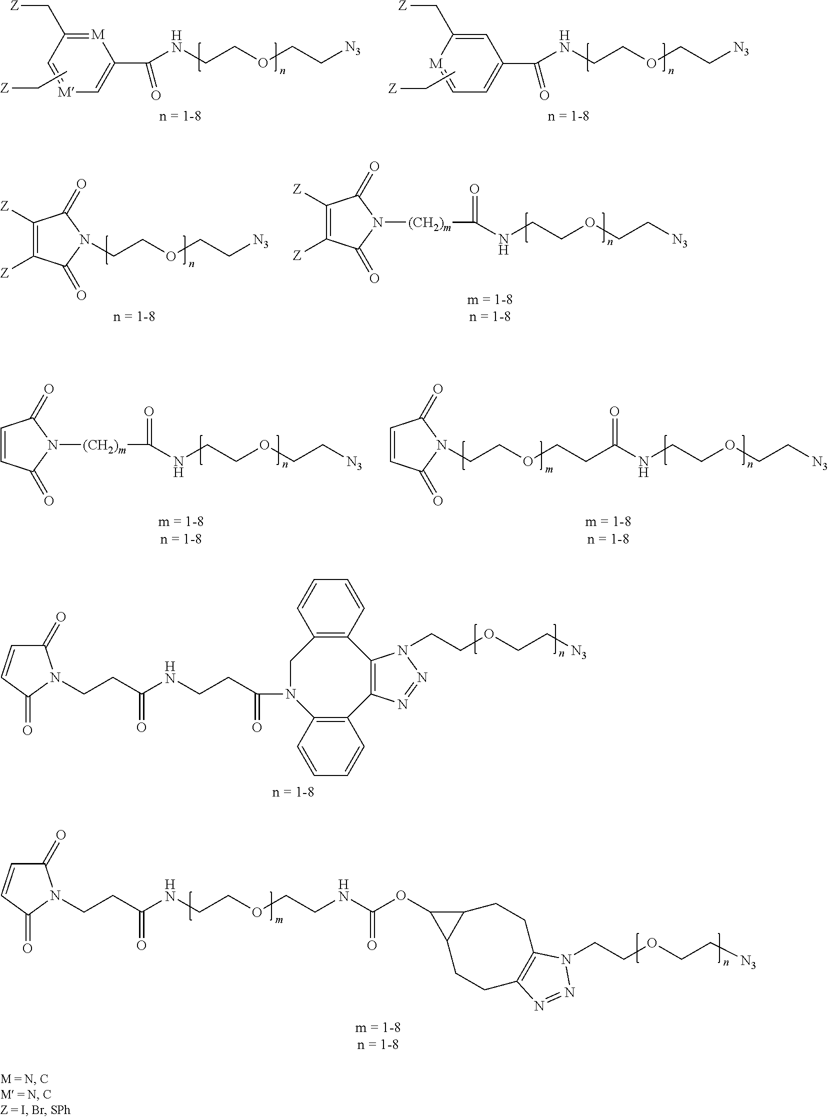
wherein N₃ is —N═N═N;
and a linked antibody B having the structure selected from the group consisting of:

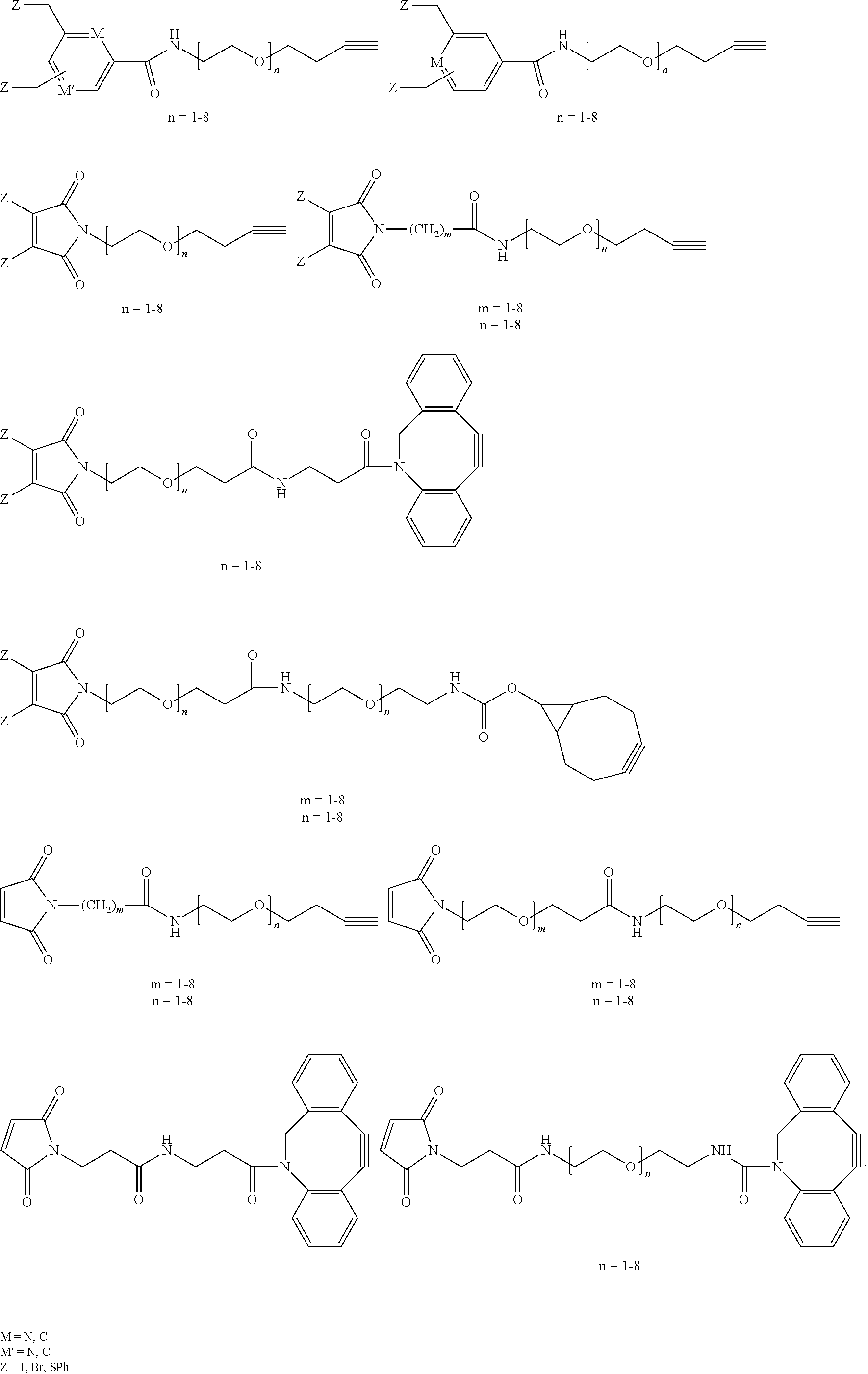

22. A chemically-locked bispecific antibody AB, comprising a linked half-antibody A linked to: 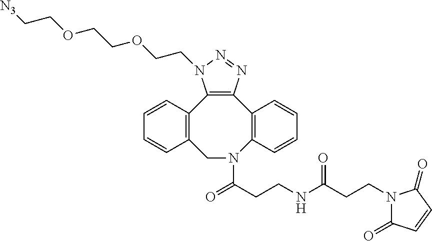
wherein N₃ is —N═N═N;
is joined to a linked antibody B linked to:

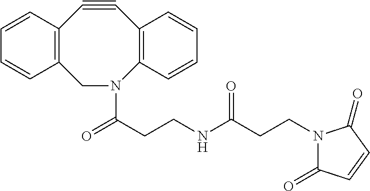 [SMILES: O=C(CCN1C(=O)C=CC1=O)NCCC(=O)N1Cc2ccccc2C#Cc2ccccc21]

23. A chemically-locked bispecific antibody “AB” or “BA” from IgG class antibody “A” and IgG class antibody “B” comprising a half-antibody A linked to a structure selected from the group consisting of: 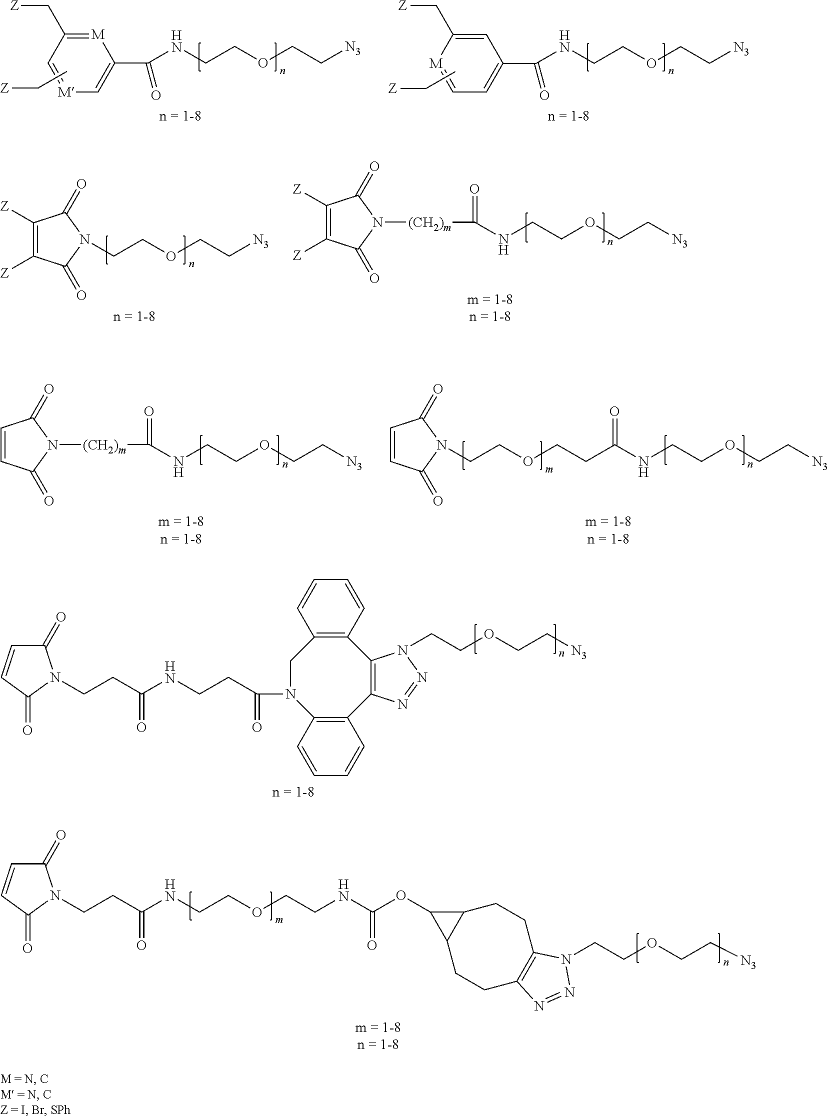
wherein N₃ is —N═N═N;
joined to a half-antibody B linked to a structure selected from the group consisting of:

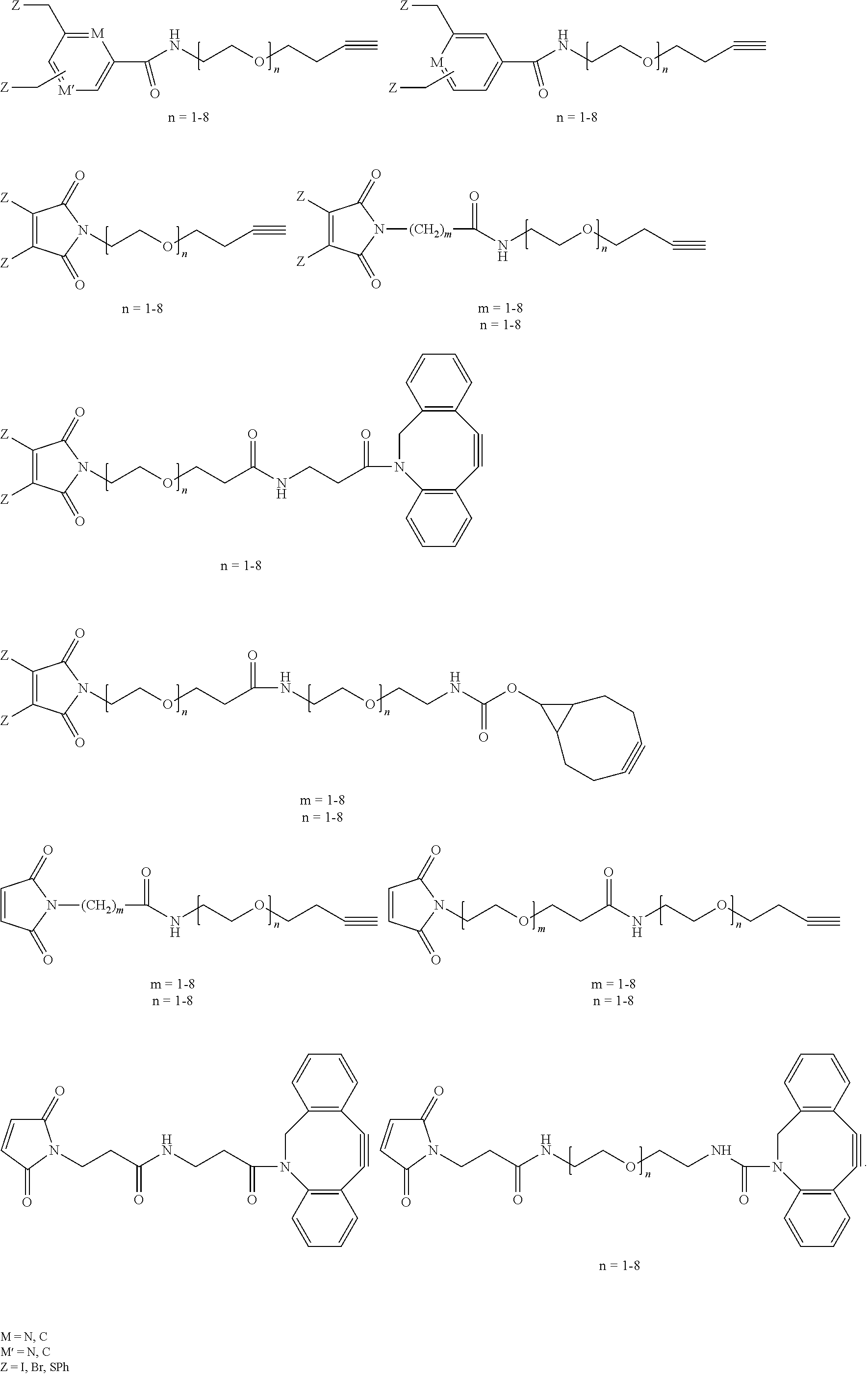

25. A bi-specific antibody comprising:
(a) a first antibody fragment A′, comprising a single heavy chain and light chain from an antibody A, wherein the single heavy chain has one or more reactive thiol groups;
(b) a second antibody fragment B′, comprising single heavy chain and light chain from an antibody B, wherein the single heavy chain has one or more reactive thiol groups;
wherein, said first and second antibody fragments are covalently linked through a 1,2,3-triazole formed by a cyloaddition reaction of an azide, attached through a linker to a reactive thiol on said first antibody fragment, and an alkyne, attached through a linker to a reactive thiol on said second antibody fragment.

26. The bi-specific antibody according to claim 25, wherein said fragment A′ and B′ are derived from IgG1 or IgG4 immunoglobulins.

27. An antibody fragment covalently bonded to a linker, the linker comprising a C₈ ring having a —C≡C— bond capable of undergoing a cyloaddition reaction with an azide.
